(19) United States
(12) Patent Application Publication
(10) Pub. No.: US 2024/0182489 A1
(43) Pub. Date: Jun. 6, 2024

(54) New 5,6,7,8-Tetrahydropyrido[4',3':4,5]thieno[2,3-d]pyrimidin-4(3H)-one Derivatives as Sigma Ligands

(71) Applicant: ACONDICIONAMIENTO TARRASENSE, TERRASSA BARCELONA) (ES)

(72) Inventors: Ute CHRISTMANN, VILADECANS (BARCELONA) (ES); Jose Luís DÍAZ-FERNÁNDEZ, MANRESA (BARCELONA) (ES)

(73) Assignee: ACONDICIONAMIENTO TARRASENSE, TERRASSA BARCELONA) (ES)

(21) Appl. No.: 18/550,958

(22) PCT Filed: Mar. 17, 2022
(86) PCT No.: PCT/EP22/57015

§ 371 Date: Sep. 16, 2023

(30) Foreign Application Priority Data
Mar. 18, 2021 (EP) 21382221.6

(51) Int. Cl. C07D 495/14; A61K 31/519

(52) U.S. Cl. CPC C07D 495/14; A61K 31/519

(57) ABSTRACT

The present invention relates to new 5,6,7,8-tetrahydropyrido[4′,3′:4,5]thieno[2,3d] pyrimidin-4(3H)-one derivatives as sigma ligands having a great affinity for sigma receptors, especially sigma-1 (σ₁), as well as to the process for the preparation thereof, to compositions comprising them, and to their use as medicaments.

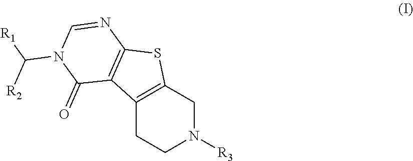

### FIELD OF THE INVENTION

The present invention relates to new 5,6,7,8-tetrahydropyrido[4′,3′:4,5]thieno[2,3d] pyrimidin-4(3H)-one derivatives as sigma ligands having a great affinity for sigma receptors, especially the sigma-1 receptor (σ₁), as well as to the process for the preparation thereof, to compositions comprising them, and to their use as medicaments.

### BACKGROUND OF THE INVENTION

The search for new therapeutic agents has been greatly aided in recent years by better understanding of the structure of proteins and other biomolecules associated with target diseases. One important class of these proteins are the sigma (σ) receptors, originally discovered in the central nervous system (CNS) of mammals in 1976 and initially related to the dysphoric, hallucinogenic and cardiac stimulant effects of opioids. Subsequent studies established a complete distinction between the a receptors binding sites and the classical opiate receptors. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease [Walker, J. M. et al., Pharmacological Reviews, (1990), 42, 355]. It has been reported that the known sigma receptor ligand rimcazole clinically shows effects in the treatment of psychosis [Snyder, S. H., Largent, B. L., J. Neuropsychiatry, (1989), 1, 7]. The sigma binding sites have preferential affinity for the dextrorotatory isomers of certain opiate benzomorphans, such as (+)-SKF-10047, (+)-cyclazocine, and (+)-pentazocine and also for some narcoleptics such as haloperidol. The sigma receptor has two subtypes that were initially discriminated by stereoselective isomers of these pharmacoactive drugs. (+)-SKF-10047 has nanomolar affinity for the sigma-1 (σ₁) site, and has micromolar affinity for the sigma-2 (σ₂) site. Haloperidol has similar affinities for both subtypes.

The σ₁ receptor is expressed in numerous adult mammal tissues (e.g. central nervous system, ovary, testicle, placenta, adrenal gland, spleen, liver, kidney, gastrointestinal tract) as well as in embryo development from its earliest stages, and is apparently involved in a large number of physiological functions. Its high affinity for various pharmaceuticals has been described, such as for (+)-SKF-10047, (+)-pentazocine, haloperidol and rimcazole, among others, known ligands with analgesic, anxiolytic, antidepressive, antiamnesic, antipsychotic and neuroprotective activity. Hence, the σ₁ receptor has possible physiological roles in processes related to analgesia, anxiety, addiction, amnesia, depression, schizophrenia, stress, neuroprotection and psychosis [Walker, J. M. et al., Pharmacological Reviews, (1990), 42, 355; Kaiser, C. et al., Neurotransmissions, (1991), 7 (1), 1-5; Bowen, W. D., Pharmaceutica Acta Helvetiae, (2000), 74, 211-218].

The σ₁ receptor is a ligand-regulated chaperone of 223 amino acids and 25 kDa cloned in 1996 and crystallized twenty years later [Hanner, M. et al., Proc. Natl. Acad. Sci. USA, (1996), 93, 8072-8077; Su, T. P. et al., Trends Pharmacol. Sci., (2010), 31, 557-566; Schmidt, H. R. et al., Nature, (2016), 532, 527-530]. Residing primarily in the interface between the endoplasmic reticulum (ER) and mitochondrion, referred to as the mitochondria-associated membrane (MAM), it can translocate to the plasma membrane or ER-membrane and regulate the activity of other proteins by modulating N-methyl-D-aspartic (NMDA) receptors and several ion channels [Monnet, F. P. et al., Eur. J. Pharmacol., (1990), 179, 441-445; Cheng, Z. X. et al., Exp. Neurol., (2010), 210, 128-136]. Owing to the role played by the σ₁R in modulating pain-related hypersensitivity and sensitization phenomena, σ₁R antagonists have been also proposed for the treatment of neuropathic pain [Drews, E. et al., Pain, 2009, 145, 269-270; De la Puente, B. et al., Pain (2009), 145, 294-303; Diaz, J. L. et al., J. Med. Chem., (2012), 55, 8211-8224; Romero et al., Brit. J. Pharm., (2012), 166, 2289-2306; Merlos, M. et al., Adv. Exp. Med. Biol., (2017), 964, 85-107]. Additionally, the σ₁ receptor has been known to modulate opioid analgesia, and the relationship between the μ-opioid and σ₁ receptors has been shown to involve direct physical interaction, which explains why σ₁ receptor antagonists enhance the antinociceptive effect of opioids without increasing their adverse effects [Chien, C. C. et al, J. Pharmacol. Exp. Ther., (1994), 271, 1583-1590; King, M. et al, Eur. J. Pharmacol., (1997), 331, R5-6; Kim, F. J. et al., Mol. Pharmacol., (2010), 77, 695-703; Zamanillo, D. et al., Eur. J. Pharmacol., (2013), 716, 78-93].

The σ₂ receptor was initially identified by radioligand binding as a site with high affinity for di-o-tolylguanidine (DTG) and haloperidol [Hellewell, S. B. et al., Brain Res., (1990), 527, 244-253]. Two decades later, progesterone receptor membrane component 1 (PGRMC1), a cytochrome-related protein that binds directly to heme and regulates lipid and drug metabolism and hormone signaling, was proposed as the complex where resides the σ₂R binding site [Xu, J. et al., Nat. Commun., (2011), 2, 380]. Finally, in 2017, the σ₂R subtype was purified and identified as transmembrane protein-97 (TMEM97), an endoplasmic-reticulum-resident molecule implicated in cholesterol homeostasis due to its association with the lysosomal Niemann-Pick cholesterol transporter type 1 (NPC1) [Alon, A. et al., Proc. Natl. Acad. Sci. USA, (2017), 114, 7160-7165; Ebrahimi-Fakhari, D. et al., Human Molecular Genetics, (2016), 25, 3588-3599]. The role of σ₂ receptor in cholesterol pathways was known since the 1990s and recent studies published by Mach et al. on modulation of trafficking and internalization of LDL by the formation of a ternary complex between LDLR, PGRMC1 and TMEM97, reinforces this association [Moebius, F. F. et al., Trends Pharmacol. Sci., (1997), 18, 67-70; Riad, A. et al., Sci. Rep., (2018), 8, 16845].

σ₂R/TMEM97, previously known also as meningioma-associated protein, MAC30, is expressed in various normal and diseased human tissues and up-regulation in certain tumors and down-regulation in other suggested that this protein played a distinct role in human malignancies. The cloning of σ₂ receptor confirmed its overexpression in epithelial, colorectal, ovarian lung and breast cancers [Moparthi, S. B. et al., Int. J. Oncol., (2007), 30, 91-95; Yan, B. Y. et al., Chemotherapy, (2010), 56, 424-428; Zhao, Z. R.; Chemotherapy, (2011), 57, 394-401; Ding, H. et al., Asian Pac. J. Cancer Prev., (2016), 17, 2705-2710]. σ₂R/TMEM97 has a molecular weight of 18-21.5 kDa and its sequence predicts a four transmembrane domain protein with cytosolic N and C terminal [Hellewell, S. B. et al., Eur. J. Pharmacol. Mol. Pharmacol. Sect., (1994), 268, 9-18]. The potential signal transduction of σ₂ receptor is not yet understood, but it seems to modulate Ca²⁺ and K⁺ channels, and to interact with caspases, epidermal growth factor receptor (EGFR), and with mammalian target of rapamycin, mTOR, signaling pathways [Vilner, B. J. et al., J. Pharmacol. Exp. Ther., (2000), 292, 900-911; Wilke, R. A. et al., J. Biol. Chem., (1999), 274, 18387-18392; Huang, Y.-S. et al., Med. Res. Rev., (2014), 34, 532-566]. These findings would explain the apoptotic effect of some σ₂ ligands by lysosome dysfunction, reactive oxygen species (ROS) production and caspase-dependent events [Ostenfeld, M. S. et al., Autophagy, (2008), 4, 487-499; Hornick, J. R. et al., J. Exp. Clin. Cancer Res., (2012), 31, 41; Zeng, C. et al., Br. J. Cancer, (2012), 106, 693-701; Pati, M. L. et al., BMC Cancer, (2017), 17, 51].

The σ₂ receptor is involved also in dopaminergic transmission, microglia activation, and neuroprotection [Guo, L. et al., Curr. Med. Chem. (2015), 22, 989-1003]. Terada et al. published in 2018 that σ₂ ligands enhance nerve growth factor (NGF)-induced neurite outgrowth in PC12 cells [Terada, K. et al., Plos One, (2018), 13, e0209250]. The σ₂ receptor plays a key role in amyloid β (Aβ)-induced synaptotoxicity, and σ₂ receptor ligands that block the interaction of Aβ oligomers with the σ₂ receptor have been shown to be neuroprotective [Izzo, N. J. et al., Plos One, (2014), 9, e111899]. σ₂ receptor modulators improve cognitive performance in a transgenic mouse model of Alzheimer's disease (AD), and in two mouse traumatic brain injury models, and could also reduce ischemic stroke injury by enhancing glial cell survival, blocking ischemia-induced glial cell activation, and decreasing nitrosative stress [Katnik, C. et al., J. Neurochem., (2016), 139, 497-509; Yi, B. et al., J. Neurochem., (2017), 140, 561-575; Vázquez-Rosa, E. et al., ACS Chem. Neurosci., (2019), 10, 1595-1602]. The σ₂ receptor has been implicated in other neurological disorders as schizophrenia [Harvey, P. D. et al., Schizophrenia Research (2020), 215, 352-356], alcohol abuse [Scott, L. L. et al., Neuropsychopharmacology, (2018), 43, 1867-1875] and pain [Sahn, J. J. et al., ACS Chem. Neurosci., (2017), 8, 1801-1811]. Norbenzomorphan UKH-1114, a σ₂ ligand, relieved mechanical hypersensitivity in the spared nerve injury (SNI) mice model of neuropathic pain, an effect explained by the preferential expression of σ₂R/TMEM97 gene in structures involved in pain such as the dorsal root ganglion (DRG).

The σ₂ receptor requires two acidic groups (Asp29, Asp56) for ligand binding, similar to σ₁R, which requires Asp126 and Glu172. σ₁R and σ₂R might have similarities in their binding sites but not necessarily other structural similarities if their amino acid sequences are compared. As σ₁R, σ₂ receptor interacts with a wide range of signaling proteins, receptors and channels, but the question if σ₂ receptor has a primarily structural or a modulatory activity remains to be answered. Several classes of σ₂ receptor ligands have been developed since Perregaard et al., synthesized Siramesine and indole analogues in 1995 [Perregaard, J. et al., J. Med. Chem., (1995), 38, 1998-2008]: tropanes [Bowen, W. D. et al., Eur. J. Pharmacol., (1995), 278, 257-260], norbenzomorphans [Sahn, J. J. et al., ACS Med. Chem. Lett., (2017), 8, 455-460], tetrahydroisoquinolines [Sun, Y.-T. et al., Eur. J. Med. Chem., (2018), 147, 227-237] or isoindolines [Grundmana, M. et al., Alzheimer's & Dementia: Translational Research & Clinical Interventions, (2019), 5, 20-26] amongst others [Berardi, F. et al., J. Med. Chem., (2004), 47, 2308-2317]. Many of these ligands present a lack of selectivity over serotoninergic receptors but mainly, there is a difficulty to reach a high selectivity over σ₁. Several σ₁-selective ligands are available, but ligands with high selectivity for σ₂ over σ₁ are relatively scarce. A significant challenge for the study of the σ₂ receptor is the paucity of highly σ₂-selective ligands.

In view of the potential therapeutic applications of agonists or antagonists of the sigma receptor, a great effort has been directed to find selective ligands. Thus, the prior art has disclosed different sigma receptor ligands, as outlined above.

Nevertheless, there is still a need to find compounds having pharmacological activity towards the sigma receptor, being both effective, selective, and/or having good “drugability” properties, i.e. good pharmaceutical properties related to administration, distribution, metabolism and excretion.

Surprisingly, it has been observed that the new 5,6,7,8-tetrahydropyrido[4′,3′:4,5]thieno[2,3d]pyrimidin-4(3H)-one derivatives with general Formula (I) show a selective affinity for sigma receptors, in particular, for σ₁. These compounds are therefore particularly suitable as pharmacologically active agents in medicaments for the prophylaxis and/or treatment of disorders or diseases related to sigma receptors.

### SUMMARY OF THE INVENTION

The present invention discloses novel compounds with great affinity to sigma receptors which might be used for the treatment of sigma related disorders or diseases. In particular, the compounds of the invention can be useful for the treatment of pain and pain related disorders and/or CNS (Central Nervous System) disorders.

The invention is directed in a main aspect to a compound of Formula (I),

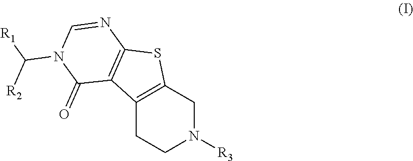

wherein R₁, R₂ and R₃ are as defined below in the detailed description.

A further aspect of the invention refers to the processes for preparation of compounds of formula (I).

It is also an aspect of the invention a pharmaceutical composition comprising a compound of formula (I).

Finally, it is an aspect of the invention a compound of formula (I) for use in therapy and more particularly for the treatment of pain and pain related conditions and/or CNS (Central Nervous System) disorders.

### DETAILED DESCRIPTION OF THE INVENTION

The invention is directed to a family of compounds, in particular, to 5,6,7,8-tetrahydropyrido[4′,3′:4,5]thieno[2,3d]pyrimidin-4(3H)-one derivatives which show a pharmacological activity towards the sigma receptors thus, solving the above problem of identifying alternative or improved pain and/or CNS treatments by offering such compounds.

The applicant has found that the problem of providing a new effective and alternative solution for treating pain and pain related disorders and/or CNS (Central Nervous System) disorders can surprisingly be solved by using compounds binding to the sigma receptors.

In a first aspect, the present invention is directed to a compound of formula (I):

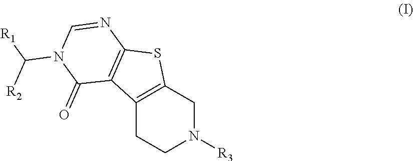

wherein:
R₁ is an aryl radical optionally substituted with one or more R₁ₐ substituents;
R₁ₐ is a hydrogen atom; a halogen atom; a branched or unbranched C₁₋₆ alkyl radical; —CN; —OR′, where R′ is hydrogen or an unsubstituted C₁₋₆ alkyl; or an optionally substituted heterocyclyl group containing one or more heteroatoms selected from N, O or S.
R₂ is a branched or unbranched C₁₋₆ alkyl radical; or a C₁₋₆ haloalkyl radical;
R₃ is a hydrogen atom; or a branched or unbranched C₁₋₆ alkyl radical;
with the proviso that when R₁ is an unsubstituted phenyl group, R₃ is not unsubstituted methyl; and with the proviso that when R₃ is a hydrogen atom, R₁ is not a phenyl group substituted with a pyrazole radical;
   wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

For the sake of clarity the expression “a compound according to formula (I), wherein R₁, R₁ₐ, R₂ and R₃ are as defined below in the detailed description” would (just like the expression “a compound of formula (I) as defined in the claims) refer to “a compound according to formula (I)”, wherein the definitions of the respective substituents R₁ etc. (also from the cited claims) are applied.

For clarity purposes, all groups and definitions described in the present description and referring to compounds of formula (I), also apply to all intermediates of synthesis.

“Halogen” or “halo” as referred in the present invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

“C₁₋₆ alkyl”, as referred to in the present invention, are saturated aliphatic radicals. They may be unbranched (linear) or branched and are optionally substituted. C₁₋₆₋alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl, 1-methylpentyl. The most preferred alkyl radical are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, are optionally mono- or polysubstituted by substitutents independently selected from a halogen, branched or unbranched C₁₋₆-alkoxy, branched or unbranched C₁₋₆-alkyl, C₁₋₆₋haloalcoxy, C₁₋₆-haloalkyl, trihaloalkyl or a hydroxyl group.

“C₁₋₆ alkoxy” as referred to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

“C₃₋₆ Cycloalkyl” as referred to in the present invention, is understood as meaning saturated and unsaturated (but not aromatic), cyclic hydrocarbons having from 3 to 6 carbon atoms which can optionally be unsubstituted, mono- or polysubstituted. Examples for cycloalkyl radical preferably include but are not restricted to cyclopropyl, cyclobutyl, cyclopentyl, cyclohexyl. Cycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A cycloalkylalkyl group/radical C₁₋₆, as defined in the present invention, comprises a branched or unbranched, optionally at least mono-substituted alkyl chain of 1 to 6 atoms which is bonded to a cycloalklyl group, as defined above. The cycloalkylalkyl radical is bonded to the molecule through the alkyl chain. A preferred cycloalkylalkyl group/radical is a cyclopropylmethyl group or a cyclopentylpropyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

A heterocyclyl radical or group (also called heterocyclyl hereinafter) is understood as meaning 4 to 18 membered mono or fused polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or fused polycyclic heterocyclic ring system of one or more rings of which at least one aromatic ring contains one or more heteroatoms from the group consisting of nitrogen, oxygen and/or sulfur in the ring; preferably it is a 5 to 18 membered mono or fused polycyclic aromatic heterocyclic ring system of one or two rings of which at least one aromatic ring contains one or more heteroatoms selected from the group consisting of nitrogen, oxygen and/or sulfur in the ring; more preferably it is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, oxadiazole, thiophene and benzimidazole;
the non-aromatic heterocyclyl is a 4 to 18 membered mono or fused polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably it is a 4 to 18 membered mono or fused polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably it is selected from azetidine, oxetane, tetrahydrofuran, oxazepam, pyrrolidine, piperidine, piperazine, tetrahydropyran, morpholine, indoline, oxopyrrolidine, benzodioxane, especially is piperazine, benzodioxane, morpholine, tetrahydropyran, piperidine, oxopyrrolidine and pyrrolidine.

Preferably, in the context of this invention heterocyclyl is defined as a 4 to 18 membered mono or fused polycyclic ring system of one or more saturated or unsaturated rings of which at least one ring contains one or more heteroatoms selected from the group consisting of nitrogen, oxygen and/or sulfur in the ring. Preferably it is a 4 to 18 membered mono or fused polycyclic heterocyclic ring system of one or two saturated or unsaturated rings of which at least one ring contains one or more heteroatoms selected from the group consisting of nitrogen, oxygen, and sulfur in the ring. More preferably, it is a 4 to 12 membered mono or bicyclic heterocyclyl ring system containing one nitrogen atom and optionally a second heteroatom selected from nitrogen and oxygen. In another preferred embodiment of the invention, said heterocyclyl is a substituted mono or bicyclic heterocyclyl ring system.

Preferred examples of heterocyclyls include azetidine, azepane, oxetane, tetrahydrofuran, oxazepam, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole and quinazoline, 3,9-diazaspiro[5.5]undecane, 2,8-diazaspiro[4.5]decane, 2,7-diazaspiro[3.5]nonane, 2,7-diazaspiro[4.4]nonane, octahydropyrrolo[3,4-c]pyrrole, especially is pyridine, piperazine, pyrazine, indazole, benzodioxane, thiazole, benzothiazole, morpholine, tetrahydropyran, pyrazole, imidazole, piperidine, thiophene, indole, benzimidazole, pyrrolo[2,3-b]pyridine, benzoxazole, oxopyrrolidine, pyrimidine, oxazepane, pyrrolidine, azetidine, azepane, oxetane, tetrahydrofuran, 3,9-diazaspiro[5.5]undecane, 2,8-diazaspiro[4.5]decane and 2,7-diazaspiro[3.5]nonane.

An N-containing heterocyclyl is a heterocyclic ring system of one or more saturated or unsaturated rings of which at least one ring contains a nitrogen and optionally one or more further heteroatoms selected from the group consisting of nitrogen, oxygen and/or sulfur in the ring; preferably is a heterocyclic ring system of one or two saturated or unsaturated rings of which at least one ring contains a nitrogen and optionally one or more further heteroatoms selected from the group consisting of nitrogen, oxygen and/or sulfur in the ring, more preferably is selected from azetidine, azepane, oxazepam, pyrrolidine, imidazole, oxadiazole, tetrazole, azetidine, pyridine, pyrimidine, piperidine, piperazine, benzimidazole, indazole, benzothiazole, benzodiazole, morpholine, indoline, triazole, isoxazole, pyrazole, pyrrole, pyrazine, pyrrolo[2,3-b]pyridine, quinoline, quinolone, isoquinoline, tetrahydrothienopyridine, phthalazine, benzo-1,2,5-thiadiazole, indole, benzotriazole, benzoxazole oxopyrrolidine, carbazole, thiazole, 3,9-diazaspiro[5.5]undecane, 2,8-diazaspiro[4.5]decane, 2,7-diazaspiro[3.5]nonane, 2,7-diazaspiro[4.4]nonane or octahydropyrrolo[3,4-c]pyrrole.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

“Heterocycloalkyl” as referred to in the present invention, are understood as meaning saturated and unsaturated (but not aromatic), generally 5 or 6 membered cyclic hydrocarbons which can optionally be unsubstituted, mono- or polysubstituted and which have at least one heteroatom in their structure selected from N, O or S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, branched or unbranched C₁₋₆-alkyl, branched or unbranched C₁₋₆-alkoxy, C₁₋₆-haloalkoxy, C₁₋₆-haloalkyl, trihaloalkyl or a hydroxyl group. More preferably heterocycloalkyl in the context of the present invention are 5 or 6-membered ring systems optionally at least monosubstituted.

A heterocycloalkylalkyl group/radical C₁₋₆, as defined in the present invention, comprises a linear or branched, optionally at least mono-substituted alkyl chain of 1 to 6 atoms which is bonded to a cycloalklyl group, as defined above. The heterocycloalkylalkyl radical is bonded to the molecule through the alkyl chain. A preferred heterocycloalkylalkyl group/radical is a piperidinethyl group or a piperazinylmethyl group, wherein the alkyl chain is optionally branched or substituted. Preferred substituents for cycloalkylalkyl group/radical, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Aryl” as referred to in the present invention, is understood as meaning ring systems with at least one aromatic ring but without heteroatoms even in only one of the rings. These aryl radicals may optionally be mono-or polysubstituted by substitutents independently selected from a halogen atom, —CN, branched or unbranched C₁₋₆-alkyl, branched or unbranched C₁₋₆-alkoxy, C₁₋₆₋haloalcoxy, C₁₋₆-haloalkyl, a heterocyclyl group and a hydroxyl group . Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present invention is a 6-membered ring system optionally at least mono or polysubstituted.

An arylalkyl radical C₁₋₆, as defined in the present invention, comprises an unbranched or branched, optionally at least mono-substituted alkyl chain of 1 to 6 carbon atoms which is bonded to an aryl group, as defined above. The arylalkyl radical is bonded to the molecule through the alkyl chain. A preferred arylalkyl radical is a benzyl group or a phenethyl group, wherein the alkyl chain is optionally branched or substituted. Preferred substituents for arylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

“Heteroaryl” as referred to in the present invention, is understood as meaning heterocyclic ring systems which have at least one aromatic ring and contain one or more heteroatoms from the group consisting of N, O or S and may optionally be mono-or polysubstituted by substituents independently selected from a halogen atom, branched or unbranched C₁₋₆-alkyl, branched or unbranched C₁₋₆-alkoxy, C₁₋₆-haloalkoxy, C₁₋₆-haloalkyl trihaloalkyl or a hydroxyl group. Preferred examples of heteroaryls include but are not restricted to furan, benzofuran, pyrrole, pyridine, pyrimidine, pyridazine, pyrazine, quinoline, isoquinoline, phthalazine, triazole, pyrazole, isoxazole, indole, benzotriazole, benzodioxolane, benzodioxane, benzimidazole, carbazole and quinazoline. More preferably heteroaryl in the context of the present invention are 5 or 6-membered ring systems optionally at least monosubstituted.

Heteroarylalkyl group/radical C₁₋₆ as defined in the present invention, comprises a linear or branched, optionally at least mono-substituted alkyl chain of 1 to 6 carbon atoms which is bonded to an heteroaryl group, as defined above. The heteroarylalkyl radical is bonded to the molecule through the alkyl chain. A preferred heteroarylalkyl radical is a piridinylmethyl group, wherein the alkyl chain is optionally branched or substituted. Preferred substituents for heteroarylalkyl radicals, according to the present invention, are independently selected from a halogen atom, branched or unbranched C₁₋₆-alkyl, branched or unbranched C₁₋₆-alkoxy, C₁₋₆-haloalcoxy, C₁₋₆-haloalkyl, trihaloalkyl or a hydroxyl group.

The term “condensed” according to the present invention means that a ring or ring-system is attached to another ring or ring-system, whereby the terms “annulated” or “annelated” are also used by those skilled in the art to designate this kind of attachment.

The term “ring system” according to the present invention refers to a system consisting of at least one ring of connected atoms but including also systems in which two or more rings of connected atoms are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

A leaving group is a group that in a heterolytic bond cleavage keeps the electron pair of the bond. Suitable leaving groups are well known in the art and include Cl, Br, I and —O—SO₂R¹⁴, wherein R¹⁴ is F, C₁₋₄-alkyl, C₁₋₄-haloalkyl, or optionally substituted phenyl. The preferred leaving groups are Cl, Br, I, tosylate, mesylate, triflate, nonaflate and fluorosulphonate.

“Protecting group” is a group that is chemically introduced into a molecule to avoid that a certain functional group from that molecule undesirably reacts in a subsequent reaction. Protecting groups are used, among others, to obtain chemoselectivity in chemical reactions. The preferred protecting group in the context of the invention are Boc (tert-butoxycarbonyl) or Teoc (2-(trimethylsilyl)ethoxycarbonyl).

The term “salt” is to be understood as meaning any form of the active compound according to the invention in which this assumes an ionic form or is charged and is coupled with a counter-ion (a cation or anion). The definition particularly includes physiologically acceptable salts, this term must be understood as equivalent to “pharmaceutically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. This definition specifically includes in the context of this invention a salt formed by a physiologically tolerated acid, i.e. salts of a specific active compound with physiologically tolerated organic or inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid. In addition, the pharmaceutically acceptable salts may be formed with a physiologically tolerated cation, preferably inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals.

The compounds of the invention may be present in crystalline form or in amorphous form.

Any compound that is a solvate of a compound according to formula (I) defined above is understood to be also covered by the scope of the invention. Methods of solvation are generally known within the art. Suitable solvates are pharmaceutically acceptable solvates. The term “solvate” is to be understood as meaning any form of the active compound according to the invention in which this compound has attached to it via non-covalent binding another molecule (most likely a polar solvent) especially including hydrates and alcoholates, like methanolate or ethanolate.

The term “co-crystal” is to be understood as a crystalline material comprising a specific active compound with at least one additional component, usually a co-crystal former, and of which at least two of the constituents are held together by weak interactions. Weak interaction is being defined as an interaction which is neither ionic nor covalent and includes for example: hydrogen bonds, van der Waals forces, and π-π [symbol] teractions.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of general formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is a N-oxide of a compound according to the invention like a compound according to formula (I) defined above is understood to be also covered by the scope of the invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

Unless otherwise defined, all the groups above mentioned that can be substituted or unsubstituted may be substituted at one or more available positions by one or more suitable groups such as a halogen, preferably Cl or F; OR′, ═O, SR′, SOR′, SO₂R′, OSO₂R′, OSO₃R′, NO₂, NHR′, NR′R″, ═N—R′, N(R′)COR′, N(COR′)₂, N(R′)SO₂R′, N(R′)C(═NR′)N(R′)R′, N₃, CN, halogen, COR′, COOR′, OCOR′, OCOOR′, OCONHR′, OCONR′R″, CONHR′, CONR′R″, CON(R′)OR′, CON(R′)SO₂R′, PO(OR′)₂, PO(OR′)R′, PO(OR′)(N(R′)R′), C₁₋₆ alkyl, C₃₋₁₀ cycloalkyl, C₂₋₆ alkenyl, C₂₋₆ alkynyl, aryl, and heterocyclic group, wherein each of the R′ and R″ groups is independently selected from the group consisting of hydrogen, C₁₋₆ alkyl, C₃₋₁₀ cycloalkyl, C₂₋₆ alkenyl, C₂₋₆ alkynyl, aryl and heterocyclic group. Where such groups are themselves substituted, the substituents may be chosen from the foregoing list.

In a particular and preferred embodiment of the invention, R₁ is an aryl radical unsubstituted or optionally substituted with one or more R₁ₐ radicals selected from a halogen atom; a branched or unbranched C₁₋₆ alkyl radical; —CN; —OR′ where R′ is a hydrogen or an unsubstituted C₁₋₆ alkyl; and an optionally substituted heterocyclyl group containing one or more heteroatoms selected from N, O or S.

In an even more preferred embodiment R₁ is a phenyl group unsubstituted or optionally substituted with one or more R₁ₐ radicals independently selected from Cl, F, a branched or unbranched C₁₋₆ alkyl radical, —CN, —OR′ where R′ is an unsubstituted C₁₋₆ alkyl, and an oxadiazole group optionally substituted with a C₁₋₆ alkyl radical.

In a still more preferred embodiment R₁ is is a phenyl group unsubstituted or optionally substituted with one or more R₁ₐ radicals independently selected from Cl and F.

In another particular and preferred embodiment of the invention, R₂ is branched or unbranched C₁₋₆ alkyl radical; or a C₁₋₆ haloalkyl radical.

In a still more preferred embodiment R₂ is methyl; ethyl; isopropyl; or trifluoromethyl.

Yet another particular and preferred embodiment of the invention is that where R₃ is hydrogen or unbranched C₁₋₆ alkyl radical.

In an even more preferred embodiment R₃ is hydrogen.

A further particular and preferred embodiment of the invention comprises a compound of formula (I):

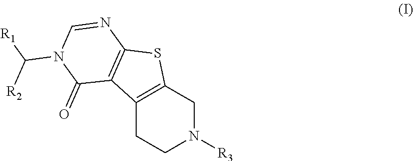

wherein
R₁ is an aryl radical unsubstituted or optionally substituted with one or more R₁ₐ radicals;
R₁ₐ is a hydrogen atom; a halogen atom; a branched or unbranched C₁₋₆ alkyl radical; —CN; —OR′ where R′ is hydrogen or an unsubstituted C₁₋₆ alkyl radical; and an optionally substituted heterocyclyl group containing one or more heteroatoms selected from N, O or S;
R₂ is a branched or unbranched C₁₋₆ alkyl radical; or a C₁₋₆ haloalkyl radical;
R₃ is a hydrogen atom; or a branched or unbranched C₁₋₆ alkyl radical;
with the proviso that when R₁ is an unsubstituted phenyl R₃ is not an unsubstituted methyl group; and with the proviso that when R₃ is a hydrogen R₁ is not a phenyl substituted with a pyrazole;
   wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A still more particular and preferred embodiment of the invention comprises a compound of formula (I):

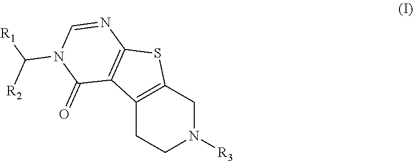

wherein:
R₁ is a phenyl radical unsubstituted or optionally substituted with one or more R₁ₐ radicals;
R₁ₐ is F, Cl, a branched or unbranched C₁₋₆ alkyl radical, —CN, —OR′ where R′ is an unsubstituted C₁₋₆ alkyl radical, and an oxadiazole group optionally substituted, preferably optionally substituted with a C₁₋₆ alkyl radical;
R₂ is a methyl, ethyl, isopropyl, or a trifluoromethyl;
R₃ is a hydrogen, a methyl or ethyl;
with the proviso that when R₁ is an unsubstituted phenyl R₃ is not an unsubstituted methyl group;
wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt, co-crystal or prodrug thereof, or a corresponding solvate thereof.

A particularly preferred embodiment of the invention is represented by compounds of formula (I) having subformulas (Ia), (Ib) or (Ic):

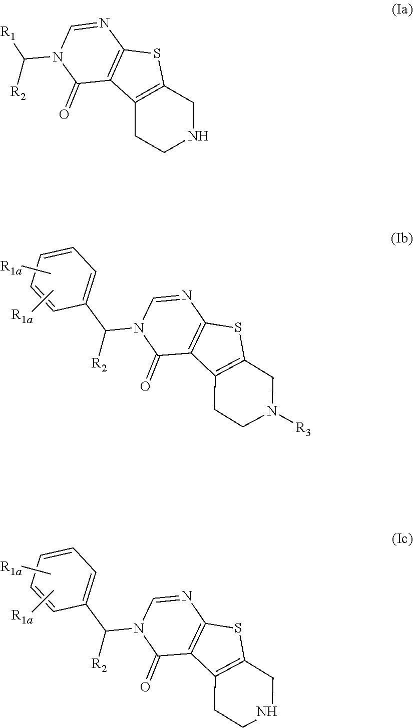

wherein R₁, R₁ₐ, R₂ and R₃ are as defined along the detailed description and claims; with the proviso that when R₁ is an unsubstituted phenyl group R₃ is not an unsubstituted methyl group; and with the proviso that when R₃ is a hydrogen R₁ is not a phenyl group substituted with a pyrazole radical;

The compounds of the present invention represented by the above described formula (I), (Ia), (Ib) or (Ic) may include enantiomers depending on the presence of chiral centers or isomers depending on the presence of double bonds (e.g. Z, E). The single stereoisomers, enantiomers or diastereoisomers and mixtures thereof fall within the scope of the present invention.

The preferred compounds of the invention are selected from:
[1] (R)-3-(1-Phenylethyl)-5,6,7,8-tetrahydropyrido[4′,3′:4,5]thieno[2,3-d]pyrimidin-4(3H)-one;
[2] S)-3-(1-Phenylethyl)-5,6,7,8-tetrahydropyrido[4′,3′:4,5]thieno[2,3-d]pyrimidin-4(3H)-one;
[3] (R)-3-(1-(4-Fluorophenyl)ethyl)-5,6,7,8-tetrahydropyrido[4′,3′:4,5]thieno[2,3-d]pyrimidin-4(3H)-one;
[4] (S)-3-(1-(4-Fluorophenyl)ethyl)-5,6,7,8-tetrahydropyrido[4′,3′:4,5]thieno[2,3-d]pyrimidin-4(3H)-one;
[5] (R)-3-(1-Phenylpropyl)-5,6,7,8-tetrahydropyrido[4′,3′:4,5]thieno[2,3-d]pyrimidin-4(3H)-one;
[6] (S)-3-(1-Phenylpropyl)-5,6,7,8-tetrahydropyrido[4′,3′:4,5]thieno[2,3-d]pyrimidin-4(3H)-one;
[7] (S)-3-(2,2,2-Trifluoro-1-phenylethyl)-5,6,7,8-tetrahydropyrido[4′,3′:4,5]thieno[2,3-d]pyrimidin-4(3H)-one;
[8] (R)-3-(2,2,2-Trifluoro-1-phenylethyl)-5,6,7,8-tetrahydropyrido[4′,3′:4,5]thieno[2,3-d]pyrimidin-4(3H)-one;
[9] (R)-3-(1-(3,4-Difluorophenyl)ethyl)-5,6,7,8-tetrahydropyrido[4′,3′:4,5]thieno[2,3-d]pyrimidin-4(3H)-one;
[10] (R)-3-(1-(2-Fluorophenyl)ethyl)-5,6,7,8-tetrahydropyrido[4′,3′:4,5]thieno[2,3-d]pyrimidin-4(3H)-one;
[11] (R)-3-(1-(3,5-Difluorophenyl)ethyl)-5,6,7,8-tetrahydropyrido[4′,3′:4,5]thieno[2,3-d]pyrimidin-4(3H)-one;
[12] (R)-3-(1-(2,4-Difluorophenyl)ethyl)-5,6,7,8-tetrahydropyrido[4′,3′:4,5]thieno[2,3-d]pyrimidin-4(3H)-one;
[13] (R)-3-(1-(4-Chlorophenyl)ethyl)-5,6,7,8-tetrahydropyrido[4′,3′:4,5]thieno[2,3-d]pyrimidin-4(3H)-one;
[14] (R)-3-(1-(4-Oxo-5,6,7,8-tetrahydropyrido[4′,3′:4,5]thieno[2,3-d]pyrimidin-3(4H)-yl)ethyl)benzonitrile;
[15] (R)-4-(1-(4-Oxo-5,6,7,8-tetrahydropyrido[4′,3′:4,5]thieno[2,3-d]pyrimidin-3(4H)-yl)ethyl)benzonitrile;
[16] (R)-3-(1-(3-Methoxyphenyl)ethyl)-5,6,7,8-tetrahydropyrido[4′,3′:4,5]thieno[2,3-d]pyrimidin-4(3H)-one;
[17] (R)-3-(1-(4-Fluorophenyl)ethyl)-5,6,7,8-tetrahydropyrido[3′,4′:4,5]thieno[2,3-d]pyrimidin-4(3H)-one;
[18] (R)-3-(2-Methyl-1-phenylpropyl)-5,6,7,8-tetrahydropyrido[4′,3′:4,5]thieno[2,3-d]pyrimidin-4(3H)-one;
[19] (S)-2-Fluoro-4-(1-(4-oxo-5,6,7,8-tetrahydropyrido[4′,3′:4,5]thieno[2,3-d]pyrimidin-3(4H)-yl)ethyl)benzonitrile;
[20] (R)-2-Fluoro-4-(1-(4-oxo-5,6,7,8-tetrahydropyrido[4′,3′:4,5]thieno[2,3-d]pyrimidin-3(4H)-yl)ethyl)benzonitrile;
[21] (R)-7-Ethyl-3-(1-phenylethyl)-5,6,7,8-tetrahydropyrido[4′,3′:4,5]thieno[2,3-d]pyrimidin-4(3H)-one;
[22] (S)-7-Ethyl-3-(1-phenylethyl)-5,6,7,8-tetrahydropyrido[4′,3′:4,5]thieno[2,3-d]pyrimidin-4(3H)-one;
[23] 3-(1-(4-Fluorophenyl)ethyl)-5,6,7,8-tetrahydropyrido[4′,3′:4,5]thieno[2,3-d]pyrimidin-4(3H)-one;
[24] 3-(1-(4-(5-Methyl-1,2,4-oxadiazol-3-yl)phenyl)ethyl)-5,6,7,8-tetrahydropyrido[4′,3′:4,5]thieno[2,3-d]pyrimidin-4(3H)-one;
[25] (S)-3-(1-(2-Chloro-4-fluorophenyl)ethyl)-5,6,7,8-tetrahydropyrido[4′,3′:4,5]thieno[2,3-d]pyrimidin-4(3H)-one;
[26] (R)-3-(1-(2-Chloro-4-fluorophenyl)ethyl)-5,6,7,8-tetrahydropyrido[4′,3′:4,5]thieno[2,3-d]pyrimidin-4(3H)-one;
[27] (S)-3-(1-(3-Chloro-5-fluorophenyl)ethyl)-5,6,7,8-tetrahydropyrido[4′,3′:4,5]thieno[2,3-d]pyrimidin-4(3H)-one;
[28] (R)-3-(1-(3-Chloro-5-fluorophenyl)ethyl)-5,6,7,8-tetrahydropyrido[4′,3′:4,5]thieno[2,3-d]pyrimidin-4(3H)-one
[29] (S)-3-(1-(5-Chloro-2-fluorophenyl)ethyl)-5,6,7,8-tetrahydropyrido[4′,3′:4,5]thieno[2,3-d]pyrimidin-4(3H)-one; and
[30] (R)-3-Fluoro-5-(1-(4-oxo-5,6,7,8-tetrahydropyrido[4′,3′:4,5]thieno[2,3-d]pyrimidin-3(4H)-yl)ethyl)benzonitrile
   or a pharmaceutical acceptable salt, stereoisomer, co-crystal, prodrug or solvate thereof.

In another aspect, the invention refers to the processes for obtaining the compounds of general formula (I). Several procedures have been developed for obtaining all the compounds of the invention, and the procedures will be explained below in methods A and B.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

### METHOD A

Method A represents a first process for synthesizing compounds according to general formula (I). Method A allows the preparation of compounds of general formula (Ia) that is compounds of general formula (I) where R₃ is hydrogen.

Thus, a process is described for the preparation of a compound of general formula (Ia):

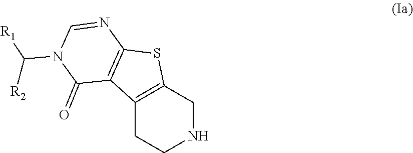

which comprises treating a compound of formula (IV) with an acid:

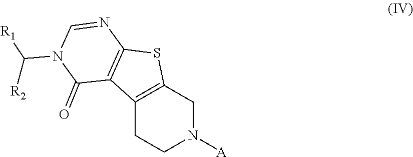

wherein R₁ and R₂ are as defined along the detailed description and the claims and A represents a protecting group, preferably a tert-butoxycarbonyl protecting group.

The compound of formula (Ia) can be prepared by treating a compound of formula (IV), where A is a protecting group, preferably a tert-butoxycarbonyl protecting group, with a suitable acid, such as trifluoroacetic acid, in a suitable solvent, such as dichloromethane, at a suitable temperature, preferably at room temperature.

### METHOD B

Method B represents a second process for synthesizing compounds according to general formula (I). Method B allows the preparation of compounds of general formula (I) where R₃ is different from hydrogen.

Therefore a process is described for the preparation of a compound of general formula (I):

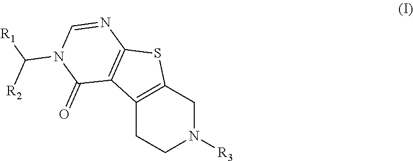

comprising the reaction between a compound of general formula (Ia):

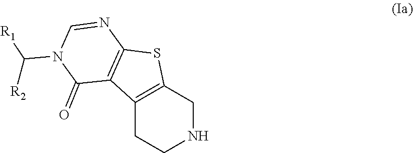

and a compound of formula (V):

LG-R₃ (V)

wherein R₁, R₂ and R₃ are as defined along the detailed description and the claims and LG is a suitable leaving group.

A compound of formula I according to method B can be prepared by reacting a compound of formula (Ia) with a suitable alkylating reagent of formula (V), in the presence of a suitable base, such as NaHCO₃, in a suitable solvent, such as ethanol, at a suitable temperature, preferably at 80° C.

In addition, a compound of formula I can be obtained in enantiopure form by resolution of a mixture of enantiomers or diastereomers of formula (I) either by chiral preparative HPLC or by crystallization of a diastereomeric salt or co-crystal. Alternatively, the resolution step can be carried out at a previous stage, using any suitable intermediate.

Scheme 1 below summarizes the synthetic routes of methods A and B.

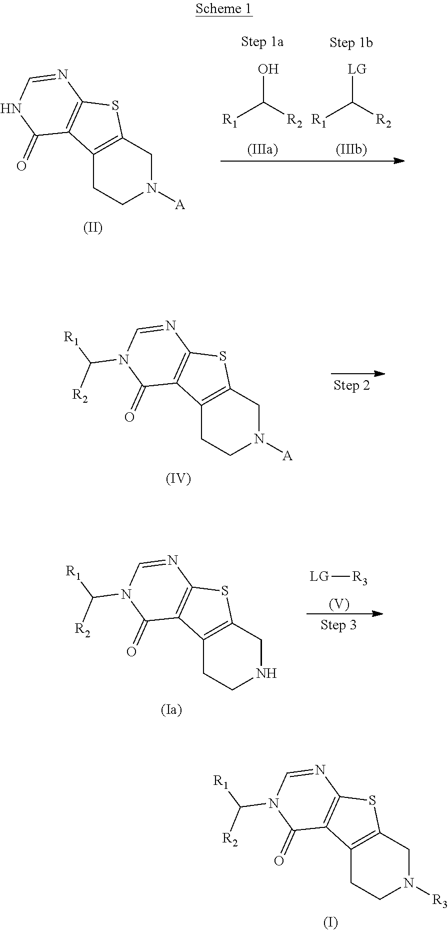

As shown in scheme 1, a compound of formula (IV) can be prepared by reacting a compound of formula (II) with a suitable alcohol of formula (IIIa) (step 1a) in the presence of a suitable dialkyl azodicarboxylate compound, such as diethyl azodicarboxylate, and a suitable phosphine, such as triphenylphosphine, in a suitable solvent, such as tetrahydrofuran, and at a suitable temperature, preferably at room temperature. Alternatively, a compound of formula (IV) can be also prepared by reacting a compound of formula (II) with a suitable alkylating agent of formula (IIIb) (step 1b) in the presence of a suitable base, such as NaH or Cs₂CO₃, in a suitable solvent, such as dimethylformamide, and at a suitable temperature, preferably between 0° C. and 100° C.

The compounds of formula II, IIIa, IIIb, and V used in the methods disclosed above are commercially available or can be synthesized following common procedures described in the literature and exemplified in the synthesis of some intermediates.

Moreover, certain compounds of the present invention can also be obtained starting from other compounds of general formula (I) by appropriate conversion reactions of functional groups, in one or several steps, using well-known reactions in organic chemistry under standard experimental conditions.

In addition, a compound of general formula (I) that shows chirality can also be obtained by resolution of a racemic compound of general formula (I) either by chiral preparative HPLC or by crystallization of a diastereomeric salt or co-crystal. Alternatively, the resolution step can be carried out at a previous stage, using any suitable intermediate.

Turning to another aspect, the invention also relates to the therapeutic use of the compounds of general formula (I). As mentioned above, compounds of general formula (I) show a strong affinity to sigma receptors, especially to sigma-1 receptors and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by sigma receptors and preferably by sigma-1 receptors. In this sense, compounds of formula (I) are suitable for the treatment and/or prophylaxis of pain, especially neuropathic pain, inflammatory pain, and chronic pain or other pain conditions involving allodynia and/or hyperalgesia, or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

The compounds of general formula (I) are especially suited for the treatment of pain, especially neuropathic pain, inflammatory pain or other pain conditions involving allodynia and/or hyperalgesia or CNS disorder or diseases, selected from the group consisting of addiction to drugs and chemical substances including cocaine, amphetamine, ethanol and nicotine, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia or paranoia.

PAIN is defined by the International Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment the compounds of the invention are used for the treatment and/or prophylaxis of neuropathic pain and more specifically for the treatment and/or prophylaxis of hyperpathia.

A related aspect of the invention refers to the use of compounds of general formula (I) for the manufacture of a medicament for the treatment and/or prophylaxis of disorders and diseases mediated by sigma receptors and more preferably by sigma-1 receptors, as explained before.

Another related aspect of the invention refers to a method for the treatment and/or prophylaxis of disorders and diseases mediated by sigma receptors and more preferably by sigma-1 receptors, as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, isomer, co-crystal, prodrug or solvate thereof, and at least a pharmaceutically acceptable carrier, additive, adjuvant or vehicle.

The pharmaceutical composition of the invention can be formulated as a medicament in different pharmaceutical forms comprising at least a compound binding to the sigma receptor and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathecal, rectal, transdermal, transmucosal or nasal administration.

The composition of the invention can be formulated for oral administration in any form preferably selected from the group consisting of tablets, drageés, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the invention can be formulated as deposits in dissolved form or in patches, for percutaneous application.

Skin applications include ointments, gels, creams, lotions, suspensions or emulsions.

The preferred form of rectal application is by means of suppositories.

In a preferred embodiment, the pharmaceutical compositions are in oral form, either solid or liquid. Suitable dose forms for oral administration may be tablets, capsules, syrups or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

In the next examples the preparation of the compounds according to the invention is disclosed.

The following abbreviations are used in the examples:
ACN: acetonitrile
Aq: aqueous
Chx: Cyclohexane
DCM: dichloromethane
DEAD: diethyl azodicarboxylate
DMF: dimethylformamide
Et₂O; diethyl ether
EtOAc; ethyl acetate
EtOH: ethanol
EX: example
h: hours
HPLC: High Performance Liquid Chromatography
MeOH: methanol
MS: mass spectrometry
Min.: minutes
Quant: quantitative
Rt.: retention time
r.t.: room temperature
Sat: saturated
TEA: triethylamine
TFA: trifluoroacetic acid
THF: tetrahydrofuran
v: volume
Wt: weight

The following methods were used to determine the HPLC-MS spectra:

### METHOD A

Column ZORBAX Extend-C18 RRHD 2.1×50 mm, 1.8 μm, temperature 35° C.; flow rate 0.61 mL/min; A: NH4HCO3 10 mM, B: ACN, C: MeOH+0.1% formic acid; gradient 0.3 min 98% A, 98% A to 0:95:5 A:B:C in 2.7 min; 0:95:5 A:B:C to 100% B in 0.1 min; isocratic 2 min 100% B.

### METHOD B

Column ZORBAX Extend-C18 RRHD 2.1×50 mm, 1.8 μm, temperature 35° C.; flow rate 0.61 mL/min; A: NH4HCO3 10 mM, B: ACN; gradient 0.3 min 98% A, 98% A to 100% B in 2.65 min; isocratic 2.05 min 100% B.

### METHOD C

Column Acquity BEH C18 2.1×50 mm, 1.7 μm, temperature 60° C.; flow rate: 1.0 mL/min; A: H₂O+0.1% TFA, B: ACN; gradient 98% A to 2% A in 2 min, 2% A to 100% B in 0.02 min, isocratic 0.5 min 100% B.

### Synthesis of Examples

### Example 1. (R)-3-(1-Phenylethyl)-5,6,7,8-tetrahydropyrido[4′,3′:4,5]thieno[2,3-d]pyrimidin-4(3H)-one

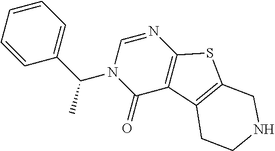 [SMILES: C[C@H](c1ccccc1)n1cnc2sc3c(c2c1=O)CCNC3]

### Step a. (R)-tert-Butyl 4-oxo-3-(1-phenylethyl)-3,4,5,6-tetrahydropyrido[4′,3′:4,5]thieno[2,3-d]pyrimidine-7(8H)-carboxylate

DEAD (0.2 mL, 1.3 mmol) was added to a solution of tert-butyl 4-oxo-3,4,5,6-tetrahydropyrido[4′,3′:4,5]thieno[2,3-d]pyrimidine-7(8H)-carboxylate (200 mg, 0.65 mmol), (S)-1-phenylethanol (119 mg, 0.97 mmol) and PPh₃ (341 mg, 1.3 mmol) in THF (10 mL) at 0° C., the solution was allowed to reach r.t. and stirred for 16 h. The reaction was quenched with water, diluted with EtOAc and washed with brine. The organic layer was dried over Na₂SO₄, filtered and the solvent was removed under vacuum. The crude product was purified by flash chromatography, silica gel, Chx:EtOAc to give the title compound (96 mg, Yield: 33%).

HPLC-MS (Method A): Rt: 2.45 min; ESI+−MS m/z: 412.2 (M+1).

### Step b. Title Compound

TFA (0.15 mL) was added to a solution of the compound obtained in step a (93 mg, 0.2 mmol) in DCM (5 mL) and the mixture was stirred at r.t. for 16 h. The reaction mixture was basified with aq. sat. NaHCO₃ and the mixture was extracted with DCM. The combined organic layers were dried over Na₂SO₄, filtered and concentrated to dryness. The crude product was purified by flash chromatography, silica gel, DCM:MeOH to give the title compound (39 mg, Yield: 60%).

HPLC-MS (Method A): Rt: 1.56 min; ESI+−MS m/z: 312.1 (M+1).

This method was used for the preparation of examples 2-18 using suitable starting materials:

| | | CHEMICAL | Rt | MS | HPLC |
|---|---|---|---|---|---|
| STRUCTURE | EX | NAME | (min) | (M + H) | Method |
| 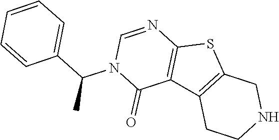 [SMILES: C[C@@H](c1ccccc1)n1cnc2sc3c(c2c1=O)CCNC3] | 2 | (S)-3-(1- Phenylethyl)- 5,6,7,8- tetrahydropyrido[4′, 3′:4,5]thieno[2,3- d]pyrimidin-4(3H)- one | 1.55 | 312.1 | A |
| 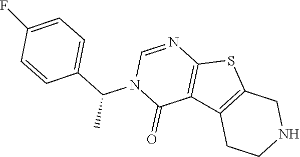 [SMILES: C[C@H](c1ccc(F)cc1)n1cnc2sc3c(c2c1=O)CCNC3] | 3 | (R)-3-(1-(4- Fluorophenyl)ethyl)- 5,6,7,8- tetrahydropyrido[4′, 3′:4,5]thieno[2,3- d]pyrimidin-4(3H)- one | 1.61 | 330.1 | A |
| 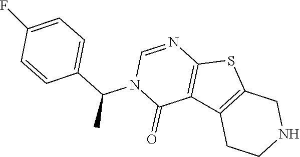 [SMILES: C[C@@H](c1ccc(F)cc1)n1cnc2sc3c(c2c1=O)CCNC3] | 4 | (S)-3-(1-(4- Fluorophenyl)ethyl)- 5,6,7,8- tetrahydropyrido[4′, 3′:4,5]thieno[2,3- d]pyrimidin-4(3H)- one | 1.58 | 330.1 | A |
| 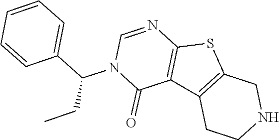 [SMILES: CC[C@H](c1ccccc1)n1cnc2sc3c(c2c1=O)CCNC3] | 5 | (R)-3-(1- Phenylpropyl)- 5,6,7,8- tetrahydropyrido[4′, 3′:4,5]thieno[2,3- d]pyrimidin-4(3H)- one | 1.67 | 325.9 | A |
| 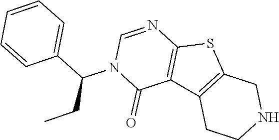 [SMILES: CC[C@@H](c1ccccc1)n1cnc2sc3c(c2c1=O)CCNC3] | 6 | (S)-3-(1- Phenylpropyl)- 5,6,7,8- tetrahydropyrido[4′, 3′:4,5]thieno[2,3- d]pyrimidin-4(3H)- one | 1.67 | 325.9 | A |
| 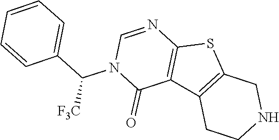 [SMILES: O=c1c2c3c(sc2ncn1[C@@H](c1ccccc1)C(F)(F)F)CNCC3] | 7 | (S)-3-(2,2,2- Trifluoro-1- phenylethyl)- 5,6,7,8- tetrahydropyrido[4′, 3′:4,5]thieno[2,3- d]pyrimidin-4(3H)- one | 1.88 | 366 | B |
| 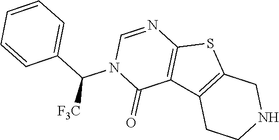 [SMILES: O=c1c2c3c(sc2ncn1[C@H](c1ccccc1)C(F)(F)F)CNCC3] | 8 | (R)-3-(2,2,2- Trifluoro-1- phenylethyl)- 5,6,7,8- tetrahydropyrido[4′, 3′:4,5]thieno[2,3- d]pyrimidin-4(3H)- one | 1.90 | 366 | B |
| 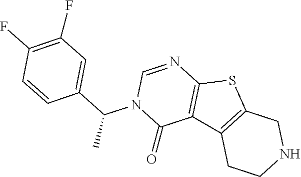 [SMILES: C[C@H](c1ccc(F)c(F)c1)n1cnc2sc3c(c2c1=O)CCNC3] | 9 | (R)-3-(1-(3,4- Difluorophenyl)ethyl)- 5,6,7,8- tetrahydropyrido[4′, 3′:4,5]thieno[2,3- d]pyrimidin-4(3H)- one | 1.62 | 347.9 | A |
| 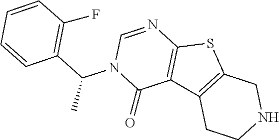 [SMILES: C[C@H](c1ccccc1F)n1cnc2sc3c(c2c1=O)CCNC3] | 10 | (R)-3-(1-(2- Fluorophenyl)ethyl)- 5,6,7,8- tetrahydropyrido[4′, 3′:4,5]thieno[2,3- d]pyrimidin-4(3H)- one | 1.56 | 330 | B |
| 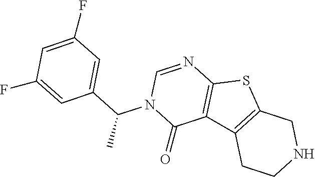 [SMILES: C[C@H](c1cc(F)cc(F)c1)n1cnc2sc3c(c2c1=O)CCNC3] | 11 | (R)-3-(1-(3,5- Difluorophenyl)ethyl)- 5,6,7,8- tetrahydropyrido[4′, 3′:4,5]thieno[2,3- d]pyrimidin-4(3H)- one | 1.65 | 348.1 | A |
| 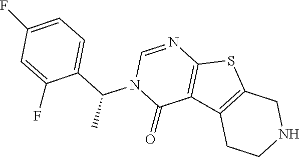 [SMILES: C[C@H](c1ccc(F)cc1F)n1cnc2sc3c(c2c1=O)CCNC3] | 12 | (R)-3-(1-(2,4- Difluorophenyl)ethyl)- 5,6,7,8- tetrahydropyrido[4′, 3′:4,5]thieno[2,3- d]pyrimidin-4(3H)- one | 1.64 | 348 | B |
| 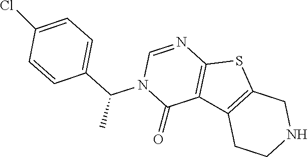 [SMILES: C[C@H](c1ccc(Cl)cc1)n1cnc2sc3c(c2c1=O)CCNC3] | 13 | (R)-3-(1-(4- Chlorophenyl)ethyl)- 5,6,7,8- tetrahydropyrido[4′, 3′:4,5]thieno[2,3- d]pyrimidin-4(3H)- one | 1.74 | 346.1 | A |
| 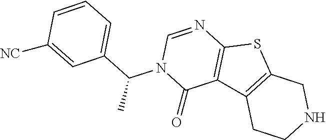 [SMILES: C[C@H](c1cccc(C#N)c1)n1cnc2sc3c(c2c1=O)CCNC3] | 14 | (R)-3-(1-(4-Oxo- 5,6,7,8- tetrahydropyrido[4′, 3′:4,5]thieno[2,3- d]pyrimidin-3(4H)- yl)ethyl)benzonitrile | 1.43 | 337.1 | A |
| 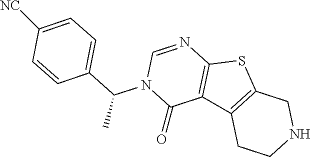 [SMILES: C[C@H](c1ccc(C#N)cc1)n1cnc2sc3c(c2c1=O)CCNC3] | 15 | (R)-4-(1-(4-Oxo- 5,6,7,8- tetrahydropyrido[4′, 3′:4,5]thieno[2,3- d]pyrimidin-3(4H)- yl)ethyl)benzonitrile | 1.42 | 337.1 | A |
| 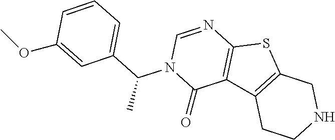 [SMILES: COc1cccc([C@@H](C)n2cnc3sc4c(c3c2=O)CCNC4)c1] | 16 | (R)-3-(1-(3- Methoxyphenyl)ethyl)- 5,6,7,8- tetrahydropyrido[4′, 3′:4,5]thieno[2,3- d]pyrimidin-4(3H)- one | 1.62 | 342 | B |
| 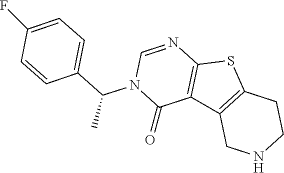 [SMILES: C[C@H](c1ccc(F)cc1)n1cnc2sc3c(c2c1=O)CNCC3] | 17 | (R)-3-(1-(4- Fluorophenyl)ethyl)- 5,6,7,8- tetrahydropyrido[3′, 4′:4,5]thieno[2,3- d]pyrimidin-4(3H)- one | 1.6 | 330 | B |
| 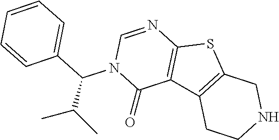 [SMILES: CC(C)[C@H](c1ccccc1)n1cnc2sc3c(c2c1=O)CCNC3] | 18 | (R)-3-(2-Methyl-1- phenylpropyl)- 5,6,7,8- tetrahydropyrido[4′, 3′:4,5]thieno[2,3- d]pyrimidin-4(3H)- one | 1.85 | 340.2 | B |

### Examples 19 and 20. (S)-2-Fluoro-4-(1-(4-oxo-5,6,7,8-tetrahydropyrido[4′,3′:4,5]thieno[2,3-d]pyrimidin-3(4H)-yl)ethyl)benzonitrile and (R)-2-fluoro-4-(1-(4-oxo-5,6,7,8-tetrahydropyrido[4′,3′:4,5]thieno[2,3-d]pyrimidin-3(4H)-yl)ethyl)benzonitrile

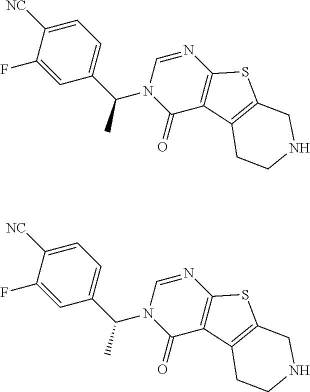 [SMILES: C[C@@H](c1ccc(C#N)c(F)c1)n1cnc2sc3c(c2c1=O)CCNC3 ; C[C@H](c1ccc(C#N)c(F)c1)n1cnc2sc3c(c2c1=O)CCNC3]

Starting from 2-fluoro-4-(1-(4-oxo-5,6,7,8-tetrahydropyrido[4′,3′:4,5]thieno[2,3-d]pyrimidin-3(4H)-yl)ethyl)benzonitrile, obtained following a similar procedure to that described in example 1, a chiral preparative HPLC separation [Column Chiralpak IA 20×250 mm, 5 μm; temperature: r.t.; eluent n-Heptane/iPrOH/Et₂NH 70/30/0.1 v/v/v; flow rate 12 mL/min; Rt1: 28.0 min; Rt2: 39.8 min] was carried out to give the title compounds.

### Example 21. (R)-7-Ethyl-3-(1-phenylethyl)-5,6,7,8-tetrahydropyrido[4′,3′:4,5]thieno[2,3-d]pyrimidin-4(3H)-one

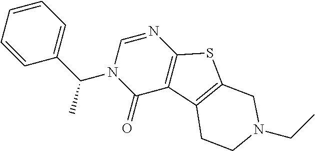 [SMILES: CCN1CCc2c(sc3ncn([C@H](C)c4ccccc4)c(=O)c23)C1]

NaHCO₃ (12 mg, 0.15 mmol) and ethyl bromide (10 μL, 0.19 mmol) were added to a solution of the compound obtained in example 1 (30 mg, 0.1 mmol) in EtOH (2 mL) and the reaction was stirred at 80° C. for 16 h. The mixture was concentrated under vacuum and the crude product was dissolved in EtOAc and washed with water and brine. The organic layer was dried over Na₂SO₄, filtered and concentrated to dryness. The crude product was purified by flash chromatography, silica gel, Chx:EtOAc to give the title compound (28 mg, Yield: 86%).

HPLC-MS (Method A): Rt: 1.97 min; ESI+−MS m/z: 340.2 (M+1).

This method was used for the preparation of example 22 using suitable starting materials:

| | | CHEMICAL | Rt | MS | HPLC |
|---|---|---|---|---|---|
| STRUCTURE | EX | NAME | (min) | (M + H) | Method |
| 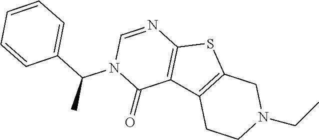 [SMILES: CCN1CCc2c(sc3ncn([C@@H](C)c4ccccc4)c(=O)c23)C1] | 22 | (S)-7-Ethyl-3-(1- phenylethyl)- 5,6,7,8- tetrahydropyrido[4′, 3′:4,5]thieno[2,3- d]pyrimidin-4(3H)- one | 2.01 | 340.1 | A |

### Example 23. 3-(1-(4-Fluorophenyl)ethyl)-5,6,7,8-tetrahydropyrido[4′,3′:4,5]thieno[2,3-d]pyrimidin-4(3H)-one

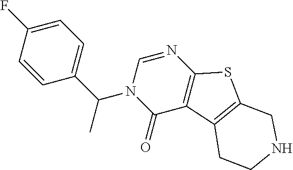 [SMILES: CC(c1ccc(F)cc1)n1cnc2sc3c(c2c1=O)CCNC3]

### Step a. tert-Butyl 3-(1-(4-fluorophenyl)ethyl)-4-oxo-3,4,5,6-tetrahydropyrido[4′,3′:4,5]thieno[2,3-d]pyrimidine-7(8H)-carboxylate

NaH (60% in mineral oil) (11.5 mg, 0.29 mmol) was added to a solution of tert-butyl 4-oxo-3,4,5,6-tetrahydropyrido[4′,3′:4,5]thieno[2,3-d]pyrimidine-7(8H)-carboxylate (80 mg, 0.26 mmol) in DMF (3 mL) and the reaction was stirred at 0° C. for 15 min. Then, 1-(1-chloroethyl)-4-fluorobenzene (49.5 mg, 0.31 mmol) was added and the mixture was stirred 1 h at r.t. and 16 h at 60° C. The mixture was concentrated under vacuum and the crude product was dissolved with an EtOAc:Et₂O (1:1) mixture and washed with water.

The organic layer was dried over Na₂SO₄, filtered and solvent was removed under vacuum. The crude product was purified by flash chromatography, silica gel, Chx:EtOAc to give the title compound (24 mg, Yield: 22%).

HPLC-MS (Method A): Rt: 2.4 min; ESI+−MS m/z: 430.2 (M+1).

### Step b. Title Compound

Starting from the compound obtained in step a (24 mg, 0.06 mmol) and following the procedure described in step b of example 1, the title compound was obtained (18 mg, Yield: 98%).

HPLC-MS (Method A): Rt: 1.58 min; ESI+−MS m/z: 330.1 (M+1).

### Example 24. 3-(1-(4-(5-Methyl-1,2,4-oxadiazol-3-yl)phenyl)ethyl)-5,6,7,8-tetrahydropyrido[4′,3′:4,5]thieno[2,3-d]pyrimidin-4(3H)-one

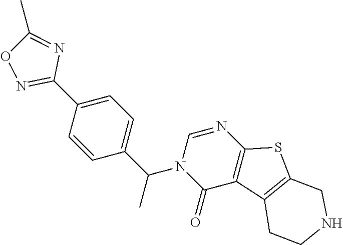 [SMILES: Cc1nc(-c2ccc(C(C)n3cnc4sc5c(c4c3=O)CCNC5)cc2)no1]

### Step a. 1-(4-(5-Methyl-1,2,4-oxadiazol-3-yl)phenyl)ethanol

A 3 M solution of methylmagnesium iodide in Et₂O (0.88 mL, 2.66 mmol) was added to a solution of 4-(5-methyl-1,2,4-oxadiazol-3-yl)benzaldehyde (250 mg, 1.33 mmol) in THF (10 mL) at −78° C., the solution was allowed to reach r.t. and stirred for 30 min. The reaction was quenched with aq. sat. NH₄Cl, diluted with an EtOAc:Et₂O (1:1) mixture and washed with brine. The organic layer was dried over Na₂SO₄, filtered and solvent was removed under vacuum to give the title compound (271 mg, Yield: 99%).

HPLC-MS (Method B): Rt: 1.42 min; ESI+−MS m/z: 205.2 (M+1).

### Step b. 3-(4-(1-Chloroethyl)phenyl)-5-methyl-1,2,4-oxadiazole

Thionyl chloride (0.11 mL, 1.5 mmol) was added to a solution of the compound obtained in step a (271 mg, 1.3 mmol) in DCM (10 mL) at 0° C. and the solution was stirred for 1 h. The reaction was diluted with EtOAc and washed with aq. sat. NaHCO₃ and brine. The organic layer was dried over Na₂SO₄, filtered and concentrated to dryness to give the title compound (270 mg, Yield: 91%).

HPLC-MS (Method A): Rt: 2.12 min; ESI+—MS m/z: 291.1 (M+1+NH₄HCO₃).

### Step c. tert-Butyl 3-(1-(4-(5-methyl-1,2,4-oxadiazol-3-yl)phenyl)ethyl)-4-oxo-3,4,5,6-tetrahydropyrido[4′,3′:4,5]thieno[2,3-d]pyrimidine-7(8H)-carboxylate

Starting from tert-butyl 4-oxo-3,4,5,6-tetrahydropyrido[4′,3′:4,5]thieno[2,3-d]pyrimidine-7(8H)-carboxylate (210 mg, 0.68 mmol) and the compound obtained in step b (182 mg, 0.82 mmol) and following the procedure described in step a of example 23, the title compound was obtained (190 mg, Yield: 56%).

HPLC-MS (Method B): 2.41 min; ESI+−MS m/z: 494.2 (M+1).

### Step d. Title Compound

Starting from the compound obtained in step c (190 mg, 0.38 mmol) and following the procedure described in step b of example 1, the title compound was obtained (130 mg, Yield: 85%).

HPLC-MS (Method A): Rt: 1.56 min, ESI+−MS m/z: 394.2 (M+1).

### Examples 25 and 26. (S)-3-(1-(2-Chloro-4-fluorophenyl)ethyl)-5,6,7,8-tetrahydropyrido[4′,3′:4,5]thieno[2,3-d]pyrimidin-4(3H)-one and (R)-3-(1-(2-chloro-4-fluorophenyl)ethyl)-5,6,7,8-tetrahydropyrido[4′,3′:4,5]thieno[2,3-d]pyrimidin-4(3H)-one

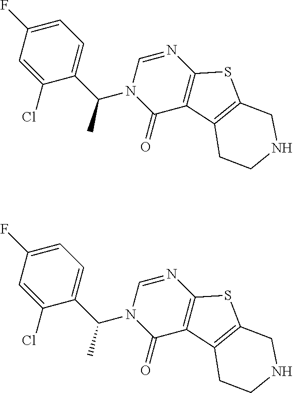 [SMILES: C[C@@H](c1ccc(F)cc1Cl)n1cnc2sc3c(c2c1=O)CCNC3 ; C[C@H](c1ccc(F)cc1Cl)n1cnc2sc3c(c2c1=O)CCNC3]

Starting from 3-(1-(2-chloro-4-fluorophenyl)ethyl)-5,6,7,8-tetrahydropyrido[4′,3′:4,5]thieno[2,3-d]pyrimidin-4(3H)-one, obtained following a similar procedure to that described in example 24 steps b-d, a chiral preparative HPLC separation [Column Chiralpak IA 20×250 mm, 5 μm; temperature: r.t.; eluent n-Heptane/iPrOH/Et₂NH 80/20/0.1 v/v/v; flow rate 10 mL/min; Rt1: 26.8 min; Rt2: 32.5 min] was carried out to give the title compounds.

### Examples 27 and 28. (S)-3-(1-(3-Chloro-5-fluorophenyl)ethyl)-5,6,7,8-tetrahydropyrido[4′,3′:4,5]thieno[2,3-d]pyrimidin-4(3H)-one and (R)-3-(1-(3-chloro-5-fluorophenyl)ethyl)-5,6,7,8-tetrahydropyrido[4′,3′:4,5]thieno[2,3-d]pyrimidin-4(3H)-one

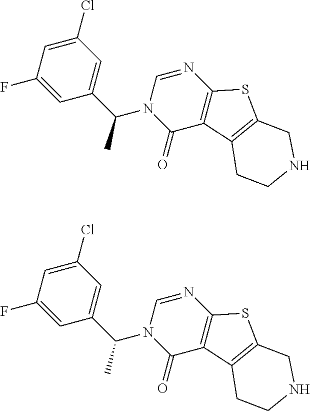 [SMILES: C[C@@H](c1cc(F)cc(Cl)c1)n1cnc2sc3c(c2c1=O)CCNC3 ; C[C@H](c1cc(F)cc(Cl)c1)n1cnc2sc3c(c2c1=O)CCNC3]

Starting from 3-(1-(3-chloro-5-fluorophenyl)ethyl)-5,6,7,8-tetrahydropyrido[4′,3′:4,5]thieno[2,3-d]pyrimidin-4(3H)-one, obtained following a similar procedure to that described in example 24 steps b-d, a chiral preparative HPLC separation [Column LUX1 21.2 mm×250 mm, 5 μm; temperature: rt; eluent: n-Heptane/EtOH/NH₃ 60/40/0.2%; Rt1: 9.01 min; Rt2: 10.26 min] was carried out to give the title compounds.

### Example 29. (S)-3-(1-(5-Chloro-2-fluorophenyl)ethyl)-5,6,7,8-tetrahydropyrido[4′,3′:4,5]thieno[2,3-d]pyrimidin-4(3H)-one

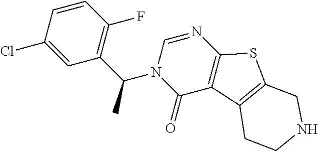 [SMILES: C[C@@H](c1cc(Cl)ccc1F)n1cnc2sc3c(c2c1=O)CCNC3]

Starting from 3-(1-(5-chloro-2-fluorophenyl)ethyl)-5,6,7,8-tetrahydropyrido[4′,3′:4,5]thieno[2,3-d]pyrimidin-4(3H)-one, obtained following a similar procedure to that described in example 24 steps b-d, a chiral preparative HPLC separation [Column Column Chiralcel OJ-H 20×250 mm, 5 μm; temperature: r.t.; eluent n-Heptane/EtOH/Et₂NH 70/30/0.1 v/v/v; flow rate 13 mL/min; Rt: 26.9 min] was carried out to give the title compound.

### Example 30. (R)-3-Fluoro-5-(1-(4-oxo-5,6,7,8-tetrahydropyrido[4′,3′:4,5]thieno[2,3-d]pyrimidin-3(4H)-yl)ethyl)benzonitrile

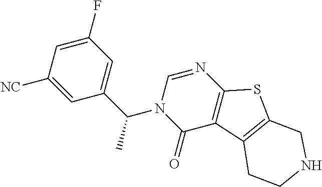 [SMILES: C[C@H](c1cc(F)cc(C#N)c1)n1cnc2sc3c(c2c1=O)CCNC3]

Starting from 3-fluoro-5-(1-(4-oxo-5,6,7,8-tetrahydropyrido[4′,3′:4,5]thieno[2,3-d]pyrimidin-3(4H)-yl)ethyl)benzonitrile, obtained following a similar procedure to that described in example 24 steps b-d, a chiral preparative HPLC separation [Column Chiralpak IB 20×250 mm, 5 μm; temperature: r.t.; eluent n-Heptane/EtOH/Et₂NH 85/15/0.05 v/v/v; flow rate 12 mL/min; Rt: 39.8 min] was carried out to give the title compound.

### Examples of Biological Activity

### Pharmacological Study

### Human σ₁ Receptor Radioligand Assay

To investigate binding properties of test compounds to human σ₁ receptor, transfected HEK-293 membranes and [³H](+)-pentazocine (Perkin Elmer, NET-1056), as the radioligand, were used. The assay was carried out with 7 μg of membrane suspension, 5 nM of [³H](+)-pentazocine in either absence or presence of either buffer or 10 μM Haloperidol for total and non-specific binding, respectively. Binding buffer contained Tris-HCl 50 mM at pH 8. Plates were incubated at 37° C. for 120 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

### Results:

As this invention is aimed at providing a compound or a chemically related series of compounds which act as ligands of the σ₁ receptor it is a very preferred embodiment in which the compounds are selected which act as ligands of the σ₁ receptor and especially compounds which have a binding expressed as Kᵢ which is preferably <1000 nM, more preferably <500 nM, even more preferably <100 nM.

The following scale as been adopted for representing the binding to the the σ₁ receptor expressed as Kᵢ:
+ Kᵢ-σ₁>=500 nM
++ Kᵢ-σ₁<500 nM
+++ Kᵢ-σ₁<100 nM

All compounds prepared in the present application exhibit binding to the σ₁ receptor, in particular, the following binding results are shown:

TABLE 1

| | Ki (nM) [symbol] |
|---|---|
| Ex | σ₁ |
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 15 | + |
| 16 | ++ |
| 17 | + |
| 18 | +++ |
| 19 | ++ |
| 20 | + |
| 21 | +++ |
| 22 | +++ |
| 23 | +++ |
| 24 | + |
| 25 | ++ |
| 26 | + |
| 27 | +++ |
| 28 | +++ |
| 29 | ++ |
| 30 | +++ |

1-15. (canceled)

16. A compound of the formula: 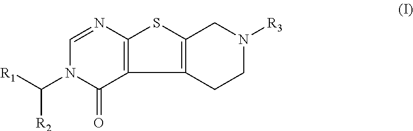
or a corresponding salt, co-crystal or prodrug thereof, or a corresponding solvate thereof, wherein
R₁ is an aryl optionally substituted with one or more R₁ₐ substituents;
each of R₁ₐ is independently a halogen atom; a branched or unbranched C₁₋₆ alkyl; —CN; an —OR′ where R′ is hydrogen or an unsubstituted C₁₋₆ alkyl; or an optionally substituted heterocyclyl group containing one or more heteroatoms selected from N, O or S;
R₂ is a branched or unbranched C₁₋₆ alkyl; or a C₁₋₆ haloalkyl;
R₃ is a hydrogen; or a branched or unbranched C₁₋₆ alkyl;
with the proviso that when R₁ is an unsubstituted phenyl, then R₃ is not methyl; and
with the proviso that when R₃ is a hydrogen, then R₁ is not a phenyl group substituted with a pyrazole.

17. The compound of claim 16, wherein said compound comprises an isomeric mixture.

18. The compound of claim 16, wherein said compound comprises a diastereomeric mixture, an enantiomeric mixture, or a racemic mixture.

19. The compound according to claim 16, wherein R₁ is aryl that is optionally substituted with one or more R₁ₐ each of which is independently selected from the group consisting of halogen; —CN; —OR′; and an optionally substituted heterocyclyl containing one or more heteroatoms selected from N, O or S, and wherein R′ is an unsubstituted C₁₋₆ alkyl.

20. The compound according to claim 16, wherein R₁ is phenyl that is optionally substituted with one or more R₁ₐ each of which is independently selected from the group consisting of F; Cl; —CN; —OR′; and oxadiazole that is optionally substituted with a C₁₋₆ alkyl, and wherein R′ is an unsubstituted C₁₋₆ alkyl.

21. The compound according to claim 16, wherein R₂ is branched or unbranched C₁₋₆ alkyl or C₁₋₆ haloalkyl.

22. The compound according to claim 16, wherein R₂ is methyl, ethyl, isopropyl, or trifluoromethyl.

23. The compound according to claim 16, wherein R₃ is hydrogen or unbranched C₁₋₆ alkyl.

24. The compound according to claim 23, wherein R₃ is methyl or ethyl.

25. The compound according to claim 16, wherein said compound is of the formula: 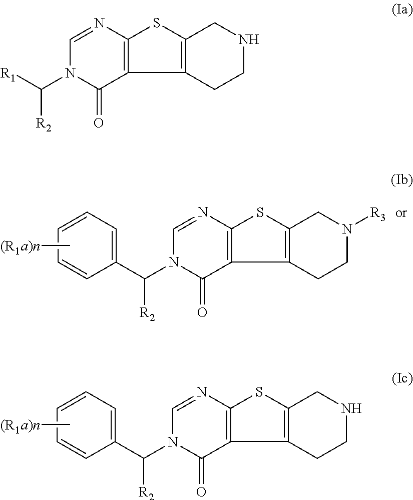
wherein R₁, R₁ₐ, R₂ and R₃ are as defined in claim 16, and n is 0, 1, or 2.

26. The compound according to claim 16 selected from the group consisting of:
(R)-3-(1-Phenylethyl)-5,6,7,8-tetrahydropyrido[4′,3′:4,5]thieno[2,3-d]pyrimidin-4(3H)-one;
(S)-3-(1-Phenylethyl)-5,6,7,8-tetrahydropyrido[4′,3′:4,5]thieno[2,3- d]pyrimidin-4(3H)-one;
(R)-3-(1-(4-Fluorophenyl)ethyl)-5,6,7,8-tetrahydropyrido[4′,3′:4,5]thieno[2,3-d]pyrimidin-4(3H)-one;
(S)-3-(1-(4-Fluorophenyl)ethyl)-5,6,7,8-tetrahydropyrido[4′,3′:4,5]thieno[2,3-d]pyrimidin-4(3H)-one;
(R)-3-(1-Phenylpropyl)-5,6,7,8-tetrahydropyrido[4′,3′:4,5]thieno[2,3-d]pyrimidin-4(3H)-one;
(S)-3-(1-Phenylpropyl)-5,6,7,8-tetrahydropyrido[4′,3′:4,5]thieno[2,3-d]pyrimidin-4(3H)-one;
(S)-3-(2,2,2-Trifluoro-1-phenylethyl)-5,6,7,8-tetrahydropyrido[4′,3′:4,5]thieno[2,3-d]pyrimidin-4(3H)-one;
(R)-3-(2,2,2-Trifluoro-1-phenylethyl)-5,6,7,8-tetrahydropyrido[4′,3′:4,5]thieno[2,3-d]pyrimidin-4(3H)-one;
(R)-3-(1-(3,4-Difluorophenyl)ethyl)-5,6,7,8-tetrahydropyrido[4′,3′:4,5]thieno[2,3-d]pyrimidin-4(3H)-one;
(R)-3-(1-(2-Fluorophenyl)ethyl)-5,6,7,8-tetrahydropyrido[4′,3′:4,5]thieno[2,3-d]pyrimidin-4(3H)-one;
(R)-3-(1-(3,5-Difluorophenyl)ethyl)-5,6,7,8-tetrahydropyrido[4′,3′:4,5]thieno[2,3-d]pyrimidin-4(3H)-one;
(R)-3-(1-(2,4-Difluorophenyl)ethyl)-5,6,7,8-tetrahydropyrido[4′,3′:4,5]thieno[2,3-d]pyrimidin-4(3H)-one;
(R)-3-(1-(4-Chlorophenyl)ethyl)-5,6,7,8-tetrahydropyrido[4′,3′:4,5]thieno[2,3-d]pyrimidin-4(3H)-one;
(R)-3-(1-(4-0xo-5,6,7,8-tetrahydropyrido[4′,3′:4,5]thieno[2,3-d]pyrimidin-3(4H)-yl)ethyl)benzonitrile;
(R)-4-(1-(4-Oxo-5,6,7,8-tetrahydropyrido[4′,3′:4,5]thieno[2,3-d]pyrimidin-3(4H)-yl)ethyl)benzonitrile;
(R)-3-(1-(3-Methoxyphenyl)ethyl)-5,6,7,8-tetrahydropyrido[4′,3′:4,5]thieno[2,3-d]pyrimidin-4(3H)-one;
(R)-3-(1-(4-Fluorophenyl)ethyl)-5,6,7,8-tetrahydropyrido[3′,4′:4,5]thieno[2,3-d]pyrimidin-4(3H)-one;
(R)-3-(2-Methyl-1-phenylpropyl)-5,6,7,8-tetrahydropyrido[4′,3′:4,5]thieno[2,3-d]pyrimidin-4(3H)-one;
(S)-2-Fluoro-4-(1-(4-oxo-5,6,7,8-tetrahydropyrido[4′,3′:4,5]thieno[2,3-d]pyrimidin-3(4H)-yl)ethyl)benzonitrile;
(R)-2-Fluoro-4-(1-(4-oxo-5,6,7,8-tetrahydropyrido[4′,3′:4,5]thieno[2,3-d]pyrimidin-3(4H)-yl)ethyl)benzonitrile;
(R)-7-Ethyl-3-(1-phenylethyl)-5,6,7,8-tetrahydropyrido[4′,3′:4,5]thieno[2,3-d]pyrimidin-4(3H)-one;
(S)-7-Ethyl-3-(1-phenylethyl)-5,6,7,8-tetrahydropyrido[4′,3′:4,5]thieno[2,3-d]pyrimidin-4(3H)-one;
3-(1-(4-Fluorophenyl)ethyl)-5,6,7,8-tetrahydropyrido[4′,3′:4,5]thieno[2,3-d]pyrimidin-4(3H)-one;
3-(1-(4-(5-Methyl-1,2,4-oxadiazol-3-yl)phenyl)ethyl)-5,6,7,8-tetrahydropyrido[4′,3′:4,5]thieno[2,3-d]pyrimidin-4(3H)-one;
(S)-3-(1-(2-Chloro-4-fluorophenyl)ethyl)-5,6,7,8-tetrahydropyrido[4′,3′:4,5]thieno[2,3-d]pyrimidin-4(3H)-one;
(R)-3-(1-(2-Chloro-4-fluorophenyl)ethyl)-5,6,7,8-tetrahydropyrido[4′,3′:4,5]thieno[2,3-d]pyrimidin-4(3H)-one;
(S)-3-(1-(3-Chloro-5-fluorophenyl)ethyl)-5,6,7,8-tetrahydropyrido[4′,3′:4,5]thieno[2,3-d]pyrimidin-4(3H)-one;
(R)-3-(1-(3-Chloro-5-fluorophenyl)ethyl)-5,6,7,8-tetrahydropyrido[4′,3′:4,5]thieno[2,3-d]pyrimidin-4(3H)-one;
(S)-3-(1-(5-Chloro-2-fluorophenyl)ethyl)-5,6,7,8-tetrahydropyrido[4′,3′:4,5]thieno[2,3-d]pyrimidin-4(3H)-one; and
(R)-3-Fluoro-5-(1-(4-oxo-5,6,7,8-tetrahydropyrido[4′,3′:4,5]thieno[2,3-d]pyrimidin-3(4H)-yl)ethyl)benzonitrile,
and a pharmaceutical acceptable salt, a stereoisomer, a co-crystal, a prodrug and a solvate thereof.

27. A process for producing a compound of the formula: 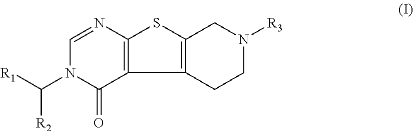
said process comprising reacting a compound of the formula: 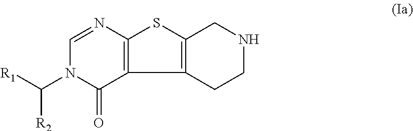
With a compound of the formula: LG-R₃ (V) under conditions sufficient to produce the compound of Formula (I),
wherein
R₁, R₂, and R₃ are as defined in claim 16, and
LG is a leaving group.

28. The process of claim 27, wherein said compound of Formula (Ia): 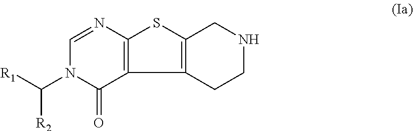
is produced by treating a compound of the formula: 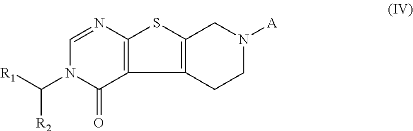
with an acid under conditions sufficient to produce the compound of Formula (Ia), wherein
R₁ and R₂ are as defined in claim 16, and
A is a protecting group.

29. The process of claim 28, wherein A is tert-butoxycarbonyl.

30. A method for treating a clinical condition associated with a sigma receptor, said method comprising administering a therapeutically effective amount of a compound of claim 16 to a subject in need of such a treatment.

31. The method of claim 30, wherein said sigma receptor is sigma-1 receptor.

32. The method of claim 30, wherein said clinical condition comprises pain or a CNS disorder or disease.

33. The method of claim 32, wherein said pain comprises neuropathic pain, inflammatory pain, chronic pain, a pain condition involving allodynia and/or hyperalgesia, or a combination thereof.

34. The method of claim 32, wherein said CNS disorder or disease is selected from the group consisting of: addiction to drugs and chemical substances, anxiety, attention-deficit-/hyperactivity disorder (ADHD), autism spectrum disorder, catalepsy, cognition disorder, learning, memory and attention deficit, depression, encephalitis, epilepsy, headache disorder, insomnia, locked-in-syndrome, meningitis, migraine, multiple sclerosis (MS), leukodystrophies, amyotrophic lateral sclerosis (ALS), myelopathy, narcolepsy, neurodegenerative disease, traumatic brain injury, Alzheimer disease, Gaucher's disease, Huntington disease, Parkinson disease, Tourette's syndrome, psychotic condition, bipolar disorder, schizophrenia and paranoia.

35. A pharmaceutical composition comprising (i) a compound of claim 16 or a pharmaceutically acceptable salt thereof, an isomer thereof, a co-crystal thereof, a prodrug thereof, or a solvate thereof; and (ii) a pharmaceutically acceptable carrier, additive, adjuvant, or vehicle, or a combination thereof.
